SEQUENCE LISTING (19 sequences)

SEQ ID NO: 1 - protein, 6 aa, Artificial Sequence
  Description of Artificial Sequence Synthetic peptide
VYSSSI

SEQ ID NO: 2 - protein, 10 aa, Artificial Sequence
  Description of Artificial Sequence Synthetic peptide
YISSYSGYTY

SEQ ID NO: 3 - protein, 6 aa, Artificial Sequence
  Description of Artificial Sequence Synthetic peptide
GRYQGM

SEQ ID NO: 4 - protein, 5 aa, Artificial Sequence
  Description of Artificial Sequence Synthetic peptide
SVSSA

SEQ ID NO: 5 - protein, 7 aa, Artificial Sequence
  Description of Artificial Sequence Synthetic peptide
SASSLYS

SEQ ID NO: 6 - protein, 6 aa, Artificial Sequence
  Description of Artificial Sequence Synthetic peptide
YAYHLI

SEQ ID NO: 7 - protein, 6 aa, Artificial Sequence
  Description of Artificial Sequence Synthetic peptide
VYSSSI

SEQ ID NO: 8 - protein, 10 aa, Artificial Sequence
  Description of Artificial Sequence Synthetic peptide
SISSYSGSTS

SEQ ID NO: 9 - protein, 9 aa, Artificial Sequence
  Description of Artificial Sequence Synthetic peptide
YGYYMSGAM

SEQ ID NO: 10 - protein, 5 aa, Artificial Sequence
  Description of Artificial Sequence Synthetic peptide
SVSSA

SEQ ID NO: 11 - protein, 7 aa, Artificial Sequence
  Description of Artificial Sequence Synthetic peptide
SASSLYS

SEQ ID NO: 12 - protein, 7 aa, Artificial Sequence
  Description of Artificial Sequence Synthetic peptide
KKAYSLI

SEQ ID NO: 13 - protein, 120 aa, Artificial Sequence
  Description of Artificial Sequence Synthetic polypeptide
EISEVQLVESGGGLVQPGGSLRLSCAASGFNVYSSSIHWVRQAPGKGLEWVAYISSYSGYTYYADSVKGRFTISADTSKNTAYLQMNSLRAEDTAVYYCARGRYQGMDYWGQGTLVTVSS

SEQ ID NO: 14 - protein, 108 aa, Artificial Sequence
  Description of Artificial Sequence Synthetic polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQYAYHLITFGQGTKVEIK

SEQ ID NO: 15 - protein, 123 aa, Artificial Sequence
  Description of Artificial Sequence Synthetic polypeptide
EISEVQLVESGGGLVQPGGSLRLSCAASGFNVYSSSIHWVRQAPGKGLEWVASISSYSGSTSYADSVKGRFTISADTSKNTAYLQMNSLRAEDTAVYYCARYGYYMSGAMDYWGQGTLVTVSS

SEQ ID NO: 16 - protein, 109 aa, Artificial Sequence
  Description of Artificial Sequence Synthetic polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQKKAYSLITFGQGTKVEIK

SEQ ID NO: 17 - protein, 15 aa, Artificial Sequence
  Description of Artificial Sequence Synthetic peptide
GGGGSGGGGSGGGGS

SEQ ID NO: 18 - protein, 15 aa, Artificial Sequence
  Description of Artificial Sequence Synthetic peptide
GGGSSGGGSSGGGSS

SEQ ID NO: 19 - protein, 4 aa, Artificial Sequence
  Description of Artificial Sequence Synthetic peptide
  See specification as filed for detailed description of substitutions and preferred embodiments
GSGS

(19) United States
(12) Patent Application Publication
(10) Pub. No.: US 2024/0327523 A1
(43) Pub. Date: Oct. 3, 2024

(54) DEPLETING MONOCLONAL ANTIBODIES AGAINST NATURAL KILLER CELLS

(71) Applicant: THE REGENTS OF THE UNIVERSITY OF CALIFORNIA, Oakland, CA (US)

(72) Inventors: LEWIS L. LANIER, Oakland, CA (US); James A. WELLS, Oakland, CA (US); Jia LIU, Oakland, CA (US); Janice ARAKAWA-HOYT, Oakland, CA (US)

(21) Appl. No.: 18/034,234

(22) PCT Filed: Nov. 15, 2021
(86) PCT No.: PCT/US2021/059383

§ 371 Date: Sep. 1, 2023

(51) Int. Cl. C07K 16/28; A61K 47/68; A61P 35/00

(52) U.S. Cl. CPC C07K 16/2851; A61K 47/6803; A61K 47/6849; A61P 35/00; C07K 16/2818; C07K 16/2827; C07K 2317/21; C07K 2317/24

(57) ABSTRACT

Anti-CD94 antibodies and their use are described.

### CROSS-REFERENCE TO RELATED PATENT APPLICATIONS

The present patent application claims benefit of priority to U.S. Provisional Patent Application No. 63/115,180, filed Nov. 18, 2020, which is incorporated by reference.

### BACKGROUND OF THE INVENTION

Groups of specific, differentiated T cells have an important role in controlling and shaping the immune response by providing a variety of immune-related functions. One of these functions is immune-mediated cell death, and it is carried out by T cells in several ways: CD8+ T cells, also known as “killer cells”, are cytotoxic-this means that they are able to directly kill virus-infected cells as well as cancer cells. CD8+ T cells are also able to utilize small signaling proteins, known as cytokines, to recruit other cells when mounting an immune response. A different population of T cells, the CD4+ T cells, function as “helper cells”. Unlike CD8+killer T cells, these CD4+ helper T cells typically function by indirectly killing cells identified as foreign: they determine if and how other parts of the immune system respond to a specific, perceived threat. There is also evidence that CD4+ T cells can directly kill virus-infected or cancer cells.

Cancer immunotherapy can involve activation and expansion of native or modified or exogenous cancer-specific T cells, which kill cancer cells by recognizing antigen targets expressed on cancer cells. The immune system, however, has ways of regulating itself to avoid overly activated responses, which may limit the immune system's ability to kill cancer cells.

### BRIEF SUMMARY OF THE INVENTION

In some embodiments, methods of enhancing a T cell response in an individual in need thereof. In some embodiments, the methods comprise administering an antibody specific for natural killer (NK) cells (a human antibody, a humanized antibody, a chimeric antibody, or a synthetic antibody) to the individual in an amount sufficient to eliminate at least some natural killer cells and thereby prevent or reduce the natural killer cell killing of activated T cells. In some embodiments, the methods comprise administering an anti-CD94 specific antibody (a human antibody, a humanized antibody, a chimeric antibody, or a synthetic antibody) to the individual in an amount sufficient to eliminate at least some natural killer cells and thereby prevent or reduce the natural killer cell killing of activated T cells. In some embodiments, the individual has cancer and activated T cells in the individual that target cancer cells in the individual.

In some embodiments, the method further comprises, before or after or simultaneous with administering the antibody (e.g., anti-CD94 specific antibody) to the individual, administering one or more checkpoint inhibitor. In some embodiments, the checkpoint inhibitor is an anti-CTLA-4, anti-PD-1, or anti-PD-L1 antibody.

In some embodiments, the anti-CD94 specific antibody comprises:
a heavy chain variable region comprising a heavy chain complementarity-determining region (CDR) 1, CDR2, and CDR3 of SEQ ID NO:1, SEQ ID NO:2, and SEQ ID NO:3, respectfully; and
a light chain variable region comprising a light chain CDR1, CDR2, and CDR3 of SEQ ID NO: 4, SEQ ID NO:5, and SEQ ID NO:6, respectfully; or
a heavy chain variable region comprising a heavy chain CDR1, CDR2, and CDR3 of SEQ ID NO: 7, SEQ ID NO:8, and SEQ ID NO:9, respectfully; and
a light chain variable region comprising a light chain CDR1, CDR2, and CDR3 of SEQ ID NO: 10, SEQ ID NO:11, and SEQ ID NO: 12, respectfully.

In some embodiments, the antibody is a chimeric antibody. In some embodiments, the chimeric antibody is a humanized antibody. In some embodiments, the antibody is a human or synthetic antibody.

In some embodiments, the anti-CD94 specific antibody comprises:
a heavy chain variable region comprising SEQ ID NO:13 and a light chain variable region comprising SEQ ID NO: 14; or
a heavy chain variable region comprising SEQ ID NO:15 and a light chain variable region comprising SEQ ID NO: 16.

In some embodiments, the antibody induces antibody-dependent cellular cytotoxicity (ADCC) or antibody-dependent phagocytosis (ADP) against natural killer cells expressing the CD94 protein. In some embodiments, the antibody is linked to a toxin to eliminate cells expressing the CD94 protein.

In some embodiments, the method comprises administering to the individual an anti-NK cell (e.g., anti-CD94 specific) antibody to the individual in an amount sufficient to transiently deplete natural killer cells. Thus, the natural killer cells will not respond to and kill allogeneic or xenogeneic cells or tissues introduced into the individual.

In some embodiments, the allogeneic or xenogeneic cell is a CAR-T cell.

In some embodiments, cells transplanted into the individual are allogeneic cells and the allogeneic cells lack HLA class I proteins. In some embodiments, the transplanted cells comprise an introduced genetic alteration that prevents or reduces CD94 expression on the transplanted cells.

In some embodiments, the individual has cancer.

In some embodiments, the anti-CD94 specific antibody comprises:
a heavy chain variable region comprising a heavy chain complementarity-determining region (CDR) 1, CDR2, and CDR3 of SEQ ID NO:1, SEQ ID NO:2, and SEQ ID NO:3, respectfully; and
a light chain variable region comprising a light chain CDR1, CDR2, and CDR3 of SEQ ID NO: 4, SEQ ID NO:5, and SEQ ID NO:6, respectfully; or
a heavy chain variable region comprising a heavy chain CDR1, CDR2, and CDR3 of SEQ ID NO: 7, SEQ ID NO:8, and SEQ ID NO:9, respectfully; and
a light chain variable region comprising a light chain CDR1, CDR2, and CDR3 of SEQ ID NO: 10, SEQ ID NO:11, and SEQ ID NO:12, respectfully.

In some embodiments, the antibody is a chimeric antibody. In some embodiments, the chimeric antibody is a humanized antibody. In some embodiments, the antibody is a human or synthetic antibody.

In some embodiments, the anti-CD94 specific antibody comprises:
a heavy chain variable region comprising SEQ ID NO:13 and a light chain variable region comprising SEQ ID NO:14; or
a heavy chain variable region comprising SEQ ID NO:15 and a light chain variable region comprising SEQ ID NO:16.

In some embodiments, the antibody induces antibody-dependent cellular cytotoxicity (ADCC) or antibody-dependent phagocytosis of natural killer cells expressing a NK-specific target protein (e.g., CD94). In some embodiments, the antibody is linked to a toxin to eliminate cells expressing the NK-specific target protein (e.g., CD94).

Also provided are methods of reducing or preventing natural killer cells from rejecting transplantation of allogeneic cells or tissue in an individual. In some embodiments, the method comprises administering to the individual, before or after the transplantation, an antibody specific for a NK-specific target protein (e.g., an anti-CD94 specific antibody) to the individual in an amount sufficient to transiently deplete natural killer cells or other cells expressing the NK-specific target protein (e.g., CD94 protein). In some embodiments, the administering of the antibody specific for the NK-specific target protein (e.g., an anti-CD94 specific antibody) before transplantation and subsequently transplanting the allogeneic or xenogeneic cells or tissue to the individual while natural killer cells or cells expressing CD94 protein are depleted.

In some embodiments, cells transplanted into the individual are allogeneic cells and the allogeneic cells lack one or more HLA class I proteins. In some embodiments, the transplanted cells comprise an introduced genetic alteration that prevents or reduces expression of the NK-specific target protein (e.g., CD94) on the transplanted cells.

In some embodiments, the anti-CD94 antibody comprises:
a heavy chain variable region comprising a heavy chain complementarity-determining region (CDR) 1, CDR2, and CDR3 of SEQ ID NO:1, SEQ ID NO:2, and SEQ ID NO:3, respectfully; and
a light chain variable region comprising a light chain CDR1, CDR2, and CDR3 of SEQ ID NO: 4, SEQ ID NO:5, and SEQ ID NO:6, respectfully; or
a heavy chain variable region comprising a heavy chain CDR1, CDR2, and CDR3 of SEQ ID NO: 7, SEQ ID NO:8, and SEQ ID NO:9, respectfully; and
a light chain variable region comprising a light chain CDR1, CDR2, and CDR3 of SEQ ID NO: 10, SEQ ID NO:11, and SEQ ID NO: 12, respectfully.

In some embodiments, the antibody is a chimeric antibody. In some embodiments, the chimeric antibody is a humanized antibody. In some embodiments, the antibody is a human or synthetic antibody.

In some embodiments, the anti-CD94 antibody comprises:
a heavy chain variable region comprising SEQ ID NO:13 and a light chain variable region comprising SEQ ID NO: 14; or
a heavy chain variable region comprising SEQ ID NO:15 and a light chain variable region comprising SEQ ID NO:16.

In some embodiments, the antibody induces antibody-dependent cellular cytotoxicity (ADCC) or antibody-dependent phagocytosis (ADP) of natural killer cells expressing the CD94 protein. In some embodiments, the antibody is linked to a toxin to eliminate cells expressing the CD94 protein.

Also provided is a monoclonal antibody that binds CD94. In some embodiments, the antibody comprises:
a heavy chain variable region comprising a heavy chain complementarity-determining region (CDR) 1, CDR2, and CDR3 of SEQ ID NO:1, SEQ ID NO:2, and SEQ ID NO:3, respectfully; and
a light chain variable region comprising a light chain CDR1, CDR2, and CDR3 of SEQ ID NO: 4, SEQ ID NO:5, and SEQ ID NO:6, respectfully; or
a heavy chain variable region comprising a heavy chain CDR1, CDR2, and CDR3 of SEQ ID NO: 7, SEQ ID NO:8, and SEQ ID NO:9, respectfully; and
a light chain variable region comprising a light chain CDR1, CDR2, and CDR3 of SEQ ID NO: 10, SEQ ID NO:11, and SEQ ID NO:12, respectfully.

In some embodiments, the antibody is a chimeric antibody. In some embodiments, the chimeric antibody is a humanized antibody.

In some embodiments, the anti-CD94 antibody comprises:
a heavy chain variable region comprising SEQ ID NO: 13 and a light chain variable region comprising SEQ ID NO: 14; or
a heavy chain variable region comprising SEQ ID NO:15 and a light chain variable region comprising SEQ ID NO:16.

### Definitions

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “antibody” as used herein includes multimeric (e.g., tetrameric) as well as single-domain antibodies, as well as antibody fragments, that retain binding specificity. An antibody as described herein can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. In some embodiments, the antibody is IgG (e.g., IgG1, IgG2, IgG3, IgG4), IgM, IgA, IgD, or IgE.

One exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

There are a number of well characterized antibody fragments. Thus, for example, pepsin digests a tetrameric antibody C-terminal to the disulfide linkages in the hinge region to produce F(ab)′2, a dimer of Fab which itself is a light chain joined to VH-CHI by a disulfide bond. The F(ab)′2 may be reduced under mild conditions to break the disulfide linkage in the hinge region thereby converting the (Fab′)2 dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody, as used herein also includes antibody fragments either produced by the modification of whole antibodies or synthesized using recombinant DNA methodologies.

As well as the specific antibody sequences provided herein, antibody substitution variants are also intended. Substitution variants can have at least one amino acid residue removed and a different residue inserted in its place. The sites of greatest interest for substitutional mutagenesis include the hypervariable regions, but framework alterations are also contemplated.

Substantial modifications in the biological properties of the antibody are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.
   Non-conservative substitutions are made by exchanging a member of one of these classes for another class.

One type of substitution that can be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical (e.g., involved in di-sulfide bond formation). Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as a Fv fragment.

Antibodies can include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. As noted above, antibodies also include single-domain antibodies, e.g., heavy chain dimers, such as antibodies from camelids.

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in which CDRs from a donor antibody are grafted onto human framework sequences or in which a non-human framework region is modified to contain human amino acid residues. Humanized antibodies may also comprise residues of donor origin in the framework sequences. The humanized antibody can also comprise at least a portion of a human immunoglobulin constant region. Humanized antibodies may also comprise residues that are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169:1119, 2002) and “resurfacing” (e.g., Staelens et al., Mol. Immunol. 43:1243, 2006; and Roguska et al., Proc. Natl. Acad. Sci USA 91:969, 1994).

As used herein, “complementarity-determining region (CDR)” refers to the three hypervariable regions (HVRs) that interrupt the four “framework” regions of a variable region. The CDRs are the primary contributors to binding to an epitope of an antigen. The CDRs of are referred to as CDR1, CDR2, and CDR3, numbered sequentially starting from the N-terminus. The term “CDR” may be used interchangeably with “HVR”.

The amino acid sequences of the CDRs and framework regions can be determined using various well-known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273 (4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1;29 (1): 207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M.J.E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g. Chothia and Lesk J. Mol. Biol. 196:901-917 (1987)).

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The term “valency” as used herein refers to the number of different binding sites of an antibody for an antigen. A monovalent antibody comprises one binding site for an antigen. A multivalent antibody comprises multiple binding sites.

The phrase “specifically (or selectively) binds” to an antigen or target or “specifically (or selectively) immunoreactive with,” when referring to a protein or peptide, refers to a binding reaction whereby the antibody binds to the antigen or target of interest. In the context of this disclosure, the antibody binds to human CD94 virus with a K_{D} that is at least 100-fold greater than its affinity for other antigens (e.g. CD20 as one example).

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, when compared and aligned for maximum correspondence over a comparison window or designated region. Alignment for purposes of determining percent amino acid sequence identity can be performed using publicly available computer software such as BLAST-2.0. The BLAST and BLAST 2.0 algorithm, are described in Altschul et al., Nuc. Acids Res. 25:3389-3402 (1977) and Altschul et al., J. Mol. Biol. 215:403-410 (1990). Thus, BLAST 2.0 can be used with the default parameters to determine percent sequence identity.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a variable domain polypeptide “corresponds to” an amino acid in a polypeptide as described herein when the residue aligns with the amino acid in a sequence when optimally aligned to the sequence. The polypeptide that is aligned to the reference sequence need not be the same length as the reference sequence.

A “conservative” substitution as used herein refers to a substitution of an amino acid such that charge, hydrophobicity, and/or size of the side group chain is maintained. Illustrative sets of amino acids that may be substituted for one another include (i) positively-charged amino acids Lys, Arg and His; (ii) negatively charged amino acids Glu and Asp; (iii) aromatic amino acids Phe, Tyr and Trp; (iv) nitrogen ring amino acids His and Trp; (v) large aliphatic nonpolar amino acids Val, Leu and Ile; (vi) slightly polar amino acids Met and Cys; (vii) small-side chain amino acids Ser, Thr, Asp, Asn, Gly, Ala, Glu, Gln and Pro; (viii) aliphatic amino acids Val, Leu, Ile, Met and Cys; and (ix) small hydroxyl amino acids Ser and Thr. Reference to the charge of an amino acid in this paragraph refers to the charge at physiological pH.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, or combinations thereof. The terms also include, but is not limited to, single-and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpin, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The terms “subject”, “patient” or “individual” are used herein interchangeably to refer to any mammal, including, but not limited to, a human. For example, the animal subject may be, a primate (e.g., a monkey, chimpanzee), a livestock animal (e.g., a horse, a cow, a sheep, a pig, or a goat), a companion animal (e.g., a dog, a cat), a laboratory test animal (e.g., a mouse, a rat, a guinea pig), or any other mammal. In some embodiments, the subject”, “patient” or “individual” is a human.

The terms “therapeutically effective dose,” “effective dose,” or “therapeutically effective amount” herein is meant a dose that produces effects for which it is administered. The exact dose and formulation will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control.

As used herein, the phrase “T cell” refers to a human lymphoid cell that expresses a T cell receptor molecule. T cells include human alpha beta (αβ) T cells and human gamma delta (γδ) T cells. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4⁺, CD8⁺, or CD4⁺ and CD8⁺, or CD4⁻, or CD8⁻. T cells can be helper cells, for example helper cells of type T_{H}1, T_{H}2, T_{H}3, T_{H}9, T_{H}17, or T_{FH}. T cells can be cytotoxic T cells. Regulatory T cells can be FOXP3⁻ or FOXP3⁻. T cells can be alpha/beta T cells or gamma/delta T cells. In some cases, the T cell is a CD4⁺D25^{hi}CD127^{lo} regulatory T cell. In some cases, the T cell is a human regulatory T cell selected from the group consisting of type 1 regulatory (Tr1), T_{H}3, CD8+CD28−, and Treg17 T-cells, or a combination or sub-population thereof. In some cases, the T cell is a FOXP3⁺ T cell. In some cases, the T cell is a CD4⁺CD25^{lo}CD127^{hi} effector T cell. In some cases, the T cell is a CD4⁺CD25^{lo}CD127^{hi}CD45RA^{hi}CD45RO⁻ naïve T cell. A T cell can be a T cell that has been genetically manipulated. In some cases, the T cell has a recombinant (e.g., heterologous) T cell receptor.

Natural killer cells, also known as NK cells, are a type of cytotoxic lymphocyte involved in the innate immune system. NK cells can be identified by the presence of CD56 and the absence of CD3 (CD56+,CD3−). See, e.g., Pfefferle A, et al., (2020). “Deciphering Natural Killer Cell Homeostasis”. Frontiers in Immunology. 11:812; Schmidt S, et al., (2018). “Natural killer cells as a therapeutic tool for infectious diseases—current status and future perspectives”. Oncotarget. 9 (29): 20891-20907. CD94 is expressed primarily on NK cells (see, e.g., Guntauri et al., Immunol Res 30 (1): 29-34 (2004)) and in the context of disclosure is considered relatively NK cell-specific. Human CD94 is described in, e.g., Chang et al., Eur. J. Immonol. 25:2433-2437 (1995) Uniprot Q13241, and occurs in at least nine different isoforms (Genbank accession numbers: NM_007334.3, NM_001351062.2, NM_001351060.2, NM_001114396.3, NM_001351063.2, NM_002262.5, NR_147038.2, NR_147039.2 and NR_147040.2).

The term “pharmaceutically acceptable salts” or “pharmaceutically acceptable carrier” or “pharmaceutically acceptable excipient” is meant to include salts of the active compounds which are prepared with relatively nontoxic acids or bases, depending on the particular substituents found on the antibodies described herein. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent.

Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present disclosure.

### BRIEF DESCRIPTION OF THE DRAWINGS

FIG. 1 results from rounds of enrichment for Fab phage binding to CD94 Fc fusion protein. The extracellular domain (ECD) of human CD94 was expressed as Fc-fusion protein in mammalian cells. The Fc-fusion proteins contain a biotin-acceptor-tag at the N-terminus and a TEV proteolysis site between the ECD and Fc-domain. These tags facilitate the ‘catch-and-release’ process in phage display selection to ensure selective release of Fab phage bound to CD94 Fc-fusion proteins. The selected Fab phages were propagated in E. coli and purified, followed by another round of selection. The selection for CD94 antibody was iterated for 4 rounds. FIG. 1 shows the Fab phage enrichment ratio for round 2-4. The selection for GFP and negative control (PBS buffer only) were implemented in the same selection campaign with the CD94 Fc-fusion protein.

FIG. 2A-B display binding curves for anti-CD94 antibodies JLS002-rAB22 and JLS002-rAB23.

FIG. 3 displays results from an octet assay used to detect the affinity of IgG for the two anti-CD94 binders JLS002-rAB22 and JLS002-rAB23.

FIG. 4A displays staining of Ba/F3 transfectants expressing human CD94. FIG. 4B displays staining of Ba/F3 transfectants expressing human CD94, NKG2D, and DAP12.

FIG. 5A-B show NK cell depletion improves the efficacy of α-PD-1L checkpoint blocked treatment for RMA lymphoma growth. FIG. 5A) experimental design. FIG. 5B) Measurements of tumor volume over time. Intraperitoneal injection of α-NK1.1, α-PD-1L, and IgG2b isotype-matched control antibody at concentrations 150 μg, 250 μg, and 250 μg were injected at indicated time points. Significance is noted as * P<0.05 by Student's t test.

### DETAILED DESCRIPTION OF THE INVENTION

The inventors have determined that natural killer cells target and can kill highly activated T cells. While this can be beneficial in many instances (for example to prevent an overly-active immune response), in some embodiments, the effect is not desirable. For example, in some embodiments, it is desirable to target cancer cells or other undesirable agents in an individual with T cells. In these aspects, it can be advantageous to deplete natural killer cells in the individual to allow activated T cells (e.g., CD8⁺ effector T cells) targeting cancer cell antigens to target and kill cancer cells in the individual. Thus, in some embodiments, an anti-CD94 antibody is administered to an individual having cancer, allowing for depletion of natural killer cells, thereby providing for enhanced targeting of cancer cells in the individual by T cells that would have otherwise been removed by the natural killer cells. This method can further include administration of one or more checkpoint inhibitors. Thus, an enhanced immune response to cancer can be induced. Administration of anti-CD94 antibodies or antibodies preferentially targeting natural killer cells can in some embodiments be accompanied by, or can follow, or be followed by, administration of an antigen that activates T cells for one or more cancer antigens.

The inventors have also discovered that one can reduce an NK cell-mediated immune response to non-self-cells or tissues in an individual by (i) administering an antibody specific for an NK-specific target protein (e.g., an anti-CD94 specific antibody) to the individual in an amount sufficient to transiently deplete natural killer cells and (ii) transplanting the non-self-cells or tissues in the individual. Expression of the non-self-cells or tissues can initially occur before or after administering the antibody (e.g., the anti-CD94 antibody). Exemplary non-self-cells or tissues can include but are not limited to a chimeric antigen receptor (CAR) T cell (including but not limited to autologous or allogenic T-cells modified to express the non-self-CAR) or T-cells (including but not limited to autologous or allogenic T-cells) expressing a heterologous T-cell receptor.

In yet other embodiments, one can reduce an NK cell-mediated immune response to autologous cells reintroduced into an individual by (i) administering an anti-CD94 specific antibody or antibodies that preferentially bind to natural killer cells to the individual in an amount sufficient to transiently deplete natural killer cells and (ii) introducing cancer-specific T-cells into the individual, where the introduced cells are generated by obtaining cells from the individual, and expanding and/or enriching for cancer-specific T-cells ex vivo.

Exemplary CD94 antibodies include those that specifically bind to the extracellular domain of CD94 and optionally do not bind to NKG2A or NKG2C. The antibodies can have a sufficiently high affinity (low K_{D}) to be pharmacologically-useful. In some embodiments, the antibodies have a K_{D} for CD94 of 10⁻⁷ M or less, 10⁻⁸ M or less, 10⁻⁹ M or less, or 10⁻¹⁰ M or less, e.g., 10⁻⁸ M-10⁻¹⁰ M or 10⁻⁸ M-10⁻¹¹ M.

In some embodiments, the anti-CD94 antibody comprises: a heavy chain variable region comprising a heavy chain complementarity-determining region (CDR) 1, CDR2, and CDR3 of VYSSSI (SEQ ID NO:1), YISSYSGYTY (SEQ ID NO:2), and GRYQGM (SEQ ID NO: 3), respectfully; and a light chain variable region comprising a light chain CDR1, CDR2, and CDR3 of SVSSA (SEQ ID NO:4), SASSLYS (SEQ ID NO:5), and YAYHLI (SEQ ID NO:6), respectfully. In some embodiments, the anti-CD94 antibody comprises: a heavy chain variable region comprising

EISEVOLVESGGGLVQPGGSLRLSCAASGFNVYSSSIHWVRQAPGKGLEWVAYISSYSG YTYYADSVKGRFTISADTSKNTAYLQMNSLRAEDTAVYYCARGRYQGMDYWGQGTL VTVSS (SEQ ID NO:13) and a light chain variable region comprising

### SDIQMTQSPSSLSASVGDRVTITCRASQSVSSAVAWYQQKPGKAPKLLIYSASSLYSGVP SRFSGSRSGTDFTLTISSLQPEDFATYYCQQYAYHLITFGQGTKVEIK (SEQ ID NO:14).

In some embodiments, the anti-CD94 antibody comprises: a heavy chain variable region comprising a heavy chain CDR1, CDR2, and CDR3 of VYSSSI (SEQ ID NO:7), SISSYSGSTS (SEQ ID NO:8), and YGYYMSGAM (SEQ ID NO:9), respectfully; and a light chain variable region comprising a light chain CDR1, CDR2, and CDR3 of SVSSA (SEQ ID NO: 10), SASSLYS (SEQ ID NO:11), and KKAYSLI (SEQ ID NO:12), respectfully. In some embodiments, the anti-CD94 antibody comprises: a heavy chain variable region comprising

EISEVOLVESGGGLVQPGGSLRLSCAASGFNVYSSSIHWVRQAPGKGLEWVASISSYSGS TSYADSVKGRFTISADTSKNTAYLQMNSLRAEDTAVYYCARYGYYMSGAMDYWGQG TLVTVSS (SEQ ID NO:15) and a light chain variable region comprising
SDIQMTQSPSSLSASVGDRVTITCRASQSVSSAVAWYQQKPGKAPKLLIYSASSLYSGVP SRFSGSRSGTDFTLTISSLQPEDFATYYCQQKKAYSLITFGQGTKVEIK (SEQ ID NO:16). Other CD94-specific antibodies can also be used.

In some embodiments, two or more different CD94 antibodies are administered, wherein the two antibodies bind to different epitopes on the surface of CD94. For example, in some embodiments, one or both of the two antibodies are selected from the antibodies described above.

One method for preparing an antibody as described herein comprises expression, in a suitable host cell or host organism or in another suitable expression system of a nucleic acid that encodes the antibody, optionally followed by isolating and/or purifying the antibody. The antibodies, including binding fragments and other derivatives thereof, can be produced by a variety of recombinant DNA techniques, including by expression in transfected cells (e.g., immortalized eukaryotic cells, such as myeloma or hybridoma cells). Suitable source cells for the DNA sequences and host cells for immunoglobulin expression and secretion can be obtained from a number of sources, such as the American Type Culture Collection (Catalogue of Cell Lines and Hybridomas, Fifth edition (1985) Rockville, Md).

In some embodiments, the antibody is a humanized antibody, i.e., an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., PNAS USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31 (3): 169-217 (1994). Techniques for humanizing antibodies are well known in the art and are described in e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534. Humanized antibodies are further described in, e.g., Winter and Milstein (1991) Nature 349:293. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well-known procedures from a variety of human cells.

In some cases, transfer of a CDR to a human framework leads to a loss of specificity for the humanized antibody. In these cases, back mutation can be introduced into the framework regions of the human portion of the antibody. Methods of making back mutations are described in, e.g., Co et al., PNAS USA 88;2269-2273 (1991) and WO 90/07861.

In some embodiments, the antibodies are antibody fragments such as Fab, F (ab′)₂, Fv, or scFv. The antibody fragments can be generated using any means known in the art including, chemical digestion (e.g., papain or pepsin) and recombinant methods. Methods for isolating and preparing recombinant nucleic acids are known to those skilled in the art (see, Sambrook et al., Molecular Cloning. A Laboratory Manual (2d ed. 1989); Ausubel et al., Current Protocols in Molecular Biology (1995)). The antibodies can be expressed in a variety of host cells, including E. coli, other bacterial hosts, yeast, and various higher eukaryotic cells such as the COS, CHO, and HeLa cells lines and myeloma cell lines.

Competitive binding assays can be used to identify antibodies that compete with an antibody described herein for specific binding to, e.g., CD94. Any of a number of competitive binding assays known in the art can be used to measure competition between two antibodies to the same antigen. Briefly, the ability of different antibodies to inhibit the binding of another antibody is tested. For example, antibodies can be differentiated by the epitope to which they bind using a sandwich ELISA assay. This is carried out by using a capture antibody to coat the surface of a well. A sub-saturating concentration of tagged-antigen is then added to the capture surface. This protein will be bound to the antibody through a specific antibody: epitope interaction. After washing a second antibody, which has been covalently linked to a detectable moiety (e.g., HRP, with the labeled antibody being defined as the detection antibody) is added to the ELISA. If this antibody recognizes the same epitope as the capture antibody it will be unable to bind to the target protein as that particular epitope will no longer be available for binding. If however this second antibody recognizes a different epitope on the target protein it will be able to bind and this binding can be detected by quantifying the level of activity (and hence antibody bound) using a relevant substrate. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

In some embodiments, the antibody (e.g., anti-CD94 antibody) is linked to a toxin, e.g., a cytotoxin such that the antibody binds to NK cells (e.g., cells expressing CD94 and kills them. Exemplary toxins can include, but are not limited to, amanitins, anthracyclines, auristatins, baccatins, calicheamicins, camptothecins, cemadotins, colchicines, colcimids., combretastatins, cryptophysins, discodermolides, duocarmycins, echinomycins, eleutherobins, epothilones, estramustines, lexitropsins, maytansinoids, netropsins, puromycins, pyrrolobenzodiazepines, rhizoxins, taxanes, tubulysins, and vinca alkaloids. Toxins can be linked to the antibody via a linker, e.g., as known in the art. See, e.g., European patent application EP3165237A1. In some embodiments, the toxin is linked to the antibody via a naturally occurring or engineered cysteine residue in the antibody, e.g., in the Fc region.

In some embodiments, an antibody can have an increased half-life due to a modification or fusion with a moiety or amino acid sequence that extends its blood half-life. In some embodiments, the antibodies are PEGylated or comprise at least one binding site for binding to a serum protein (such as serum albumin) or other moiety (and in particular at least one amino acid sequence) which increases the half-life of the antibody. Examples include but are not limited to serum albumin (such as human serum albumin), serum immunoglobulins such as IgG, or transferrin; an Fc portion (such as a human Fc) or a suitable part or fragment thereof; or one or more small proteins or peptides that can bind to serum proteins, such as, without limitation, the proteins and peptides described in WO 91/01743, WO 01/45746, WO 02/076489, WO 08/068280, WO 09/127691 and WO 11/095545.

In some embodiments, two portions of a polypeptide, e.g., linking a variable domain to another sequence (e.g., a second variable region or half-life extender or other sequence) can be directly linked to each other or can be linked to each other via one or more suitable spacers or linkers. Preferably, the linker or spacer is suitable for use in constructing proteins or polypeptides that are intended for pharmaceutical use. Some particularly preferred spacers include the spacers and linkers that are used in the art to link antibody fragments or antibody domains. For example, a linker may be a suitable amino acid sequence, and in particular amino acid sequences of between 1 and 50, preferably between 1 and 30, such as between 1 and 10 amino acid residues. Some examples of such amino acid sequences include gly-ser linkers, for example of the type (glyₓser_{y})₂, such as (for example (gly₄ser)₃ or (glysser₂)₃, as described in WO 99/42077 and the GS30, GS15, GS9 and GS7 linkers described in WO 06/040153 and WO 06/122825), as well as hinge-like regions, such as the hinge regions of naturally occurring heavy chain antibodies or similar sequences (such as described in WO 94/04678).

In some embodiments, the anti-CD94 antibody or antibodies preferentially binding to natural killer cells is administered in conjunction with checkpoint inhibitor therapy. The anti-CD94 antibody can be administered before, after, or during administration of the one or more checkpoint inhibitor. Thus, in some embodiments the checkpoint inhibitor is selected from a PD-1 inhibitor, a PD-L1 inhibitor, and a CTLA-4 inhibitor or a combination thereof. Exemplary inhibitors can include but are not limited to antibodies that bind to the immune pathway checkpoint protein in question (e.g., PD-1 or PD-L1 or CTLA-4) and prevent binding of receptor to ligand. PD-1 and CTLA-4 inhibition is discussed in, e.g., Buchbinder, and Desai, Am J Clin Oncol. 2016 February; 39 (1): 98-106. Exemplary CTLA-4 antibodies include but are not limited to Ipilimumab (trade name Yervoy™) as well as those described in, e.g., WO 2001/014424, U.S. Pat. Nos. 7,452,535; 5,811,097. Exemplary PD-1 antibodies include but are not limited to Pembrolizumab (formerly MK-3475 and lambrolizumab, trade name Keytruda™), Nivolumab (Opdivo) and Cemiplimab (Libtayo) as well as those described in, e.g., U.S. Pat. No. 8,008,449 and Zarganes-Tzitzikas, et al., Journal Expert Opinion on Therapeutic Patents Volume 26, 2016, Issue 9. Exemplary PD-L1 antibodies include but are not limited to Atezolizumab (Tecentriq), Avelumab (Bavencio), and Durvalumab (Imfinzi).

Also provided are polynucleotides (e.g., DNA) encoding the antibodies as described herein, e.g., encoding the CDRs and optionally the entire variable regions as described herein. Also provided are vectors (e.g., plasmids, viral vectors, etc.) comprising a polynucleotide sequence encoding the antibodies as descried herein, optionally wherein a promoter is operably linked to the polynucleotide for expression in a cell. Also provided are prokaryotic (e.g., E. coli) or eukaryotic cells (e.g., mammalian, human, insect, plant or yeast cells) containing the polynucleotides or vectors.

In one aspect, one or more antibody as described herein is formulated as a pharmaceutical composition, e.g., in a therapeutically effective amount using a dosing regimen suitable for treatment of cancer or for reducing or prevention an immune response against cells or tissues transplanted into the individual. The composition can be formulated for use in a variety of drug delivery systems.

The pharmaceutical compositions may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

The compositions, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like. In some embodiments they are delivered to the subject via an inhaler. Accordingly, in some embodiments, an inhaler containing a pharmaceutical formulation comprising an antibody as described herein is provided.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The compositions can also be administered as part of a prepared food or drug.

The dose administered to a patient, in the context of the present methods and compositions should be sufficient to effect a beneficial response in the subject over time. The optimal dose level for any patient will depend on a variety of factors including the efficacy of the specific modulator employed, the age, body weight, physical activity, and diet of the patient, and on a possible combination with other drugs. The size of the dose also will be determined by the existence, nature, and extent of any adverse side-effects that accompany the administration of a particular compound or vector in a particular subject.

In determining the effective amount of active components (e.g., an antibody) to be administered a physician may evaluate circulating plasma levels of the active component and its toxicity. In general, the dose equivalent of an antibody is from about 1 ng/kg to 10 mg/kg for a typical subject. Administration can be accomplished via single or divided doses.

The compositions may be administered on a regular basis (e.g., daily) for a period of time (e.g., 2, 3, 4, 5, 6, days or 1-3 weeks or more). The compositions can be administered directly to the mammalian subject to deplete natural killer cells using any route known in the art, including e.g., by injection (e.g., intravenous, intraperitoneal, subcutaneous, intramuscular, or intradermal), inhalation, or transdermal application.

Subjects receiving an antibody as described herein can include subjects who have been diagnosed with cancer. Depending on whether or not tumors can spread by invasion and metastasis, they are classified as being either benign or malignant: benign tumors are tumors that cannot spread by invasion or metastasis, i.e., they only grow locally; whereas malignant tumors are tumors that are capable of spreading by invasion and metastasis. The methods described herein are useful for the treatment of local and malignant tumors. Exemplary types of cancer include, but are not limited to: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyo sarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will-be known to one of ordinary skill in the art.

In some embodiments, a cell introduced into the individual comprises a heterologous protein expressed by a heterologous nucleic acid. Non-limiting examples of suitable methods for introducing a nucleic acid into a cell include electroporation (e.g., nucleofection), viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. In some embodiments, a polynucleotide encoding a protein is delivered to the cell by a vector. For example, in some embodiments, the vector is a viral vector. Exemplary viral vectors can include, but are not limited to, adenoviral vectors, adeno-associated viral (AAV) vectors, and lentiviral vectors.

Currently, CAR T cell products are made from autologous T cells. These CAR T cells approved for clinical use must be generated on a custom-made basis. This case-by-case autologous T cell production platform remains a significant limiting factor for large-scale clinical application due to the costly and lengthy production process. There is also an inherent risk of production failure. The individualized, custom-made autologous CAR T cell production process also posts constriction on the wide application on diverse tumor types. Therefore, universal allogeneic T cells are needed for the preparation of universal CAR T cells that can serve as the “off-the-shelf” ready-to-use therapeutic agents for large-scale clinical applications. Transient depletion of NK cells can enable engraftment of universal CAR T cells. It is particularly useful to deplete NK cells in the host if the allogeneic T cells have been engineered (e.g., by CRISPR) to abolish their expression of their HLA class I proteins-either by disabling the b2-microglobulin gene or by disabling the HLA class I heavy chain genes such as HLA-A, HLA-B, HLA-C or other polymorphic HLA class I genes, as NK cells preferentially kill HLA class I-negative cells. In some embodiments, depletion of NK cells enhances the expansion of autologous CAR T cells in a patient by deleting NK cells that can kill autologous T cells that are highly activated.

In some embodiments, following administration of the anti-NK cell antibodies (e.g., anti-CD94 antibodies), and their subsequent depletion of mature NK cells, newly arising NK cells may become tolerant to the transferred allogeneic cells or tissues, e.g., HLA class I-negative cells or tissues.

Optionally, cells introduced into the animal (for example a CAR T-cell or other T-cell) can be modified, for example but not limited to by CRISPR, to disable the CD94 (KLRD1) gene in these cells, thereby avoiding any potential toxic effect of anti-CD94 antibodies to the introduced cells in the situation where the activated CAR T cells have acquired expression of CD94.

Chimeric antigen receptors (CARs) are recombinant receptor constructs comprising an extracellular antigen-binding domain (e.g., a nanobody) joined to a transmembrane domain, and further linked to an intracellular signaling domain (e.g., an intracellular T cell signaling domain of a T cell receptor component and an intracellular domain of a costimulatory receptor such as CD28 or CD137 or others) that transduces a signal to elicit a function. In certain embodiments, immune cells (e.g., T cells or natural killer (NK) cells) are genetically modified to express CARs that comprise one or more antigen recognition domain and have the functionality of effector cells (e.g., cytotoxic and/or memory functions of T cells or NK cells).

Any transmembrane suitable for use in a CAR construct may be employed. Such transmembrane domains include, but are not limited to, all or part of the transmembrane domain of the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CDID, ITGAE, CD103, ITGAL, CDI 1a, LFA-1, ITGAM, CD11b, ITGAX, CDI 1c, ITGB 1, CD29, ITGB2, CD 18, LFA-1, ITGB7, TNFR2, DNAMI (CD226), SLAMF4 (CD244, 2B4), CD84, CD96 (Tactile), CEACAM1, CRT AM, Ly9 (CD229), CD160 (BY55), PSGL1, CD100, (SEMA4D), SLAMF6 (NTB-A, Ly108), SLAM (SLAMF1, CD150, IPO-3), BLAME, (SLAMF8), SELPLG (CD162), LTBR, PAG/Cbp, NKG2D, or NKG2C.

### EXAMPLE

### Example 1

CD94 is a membrane protein on NK cell surface, being upregulated in activated NK cells. To generate antibodies against CD94, we expressed the ectodomain of CD94 as a Fc-fusion protein in mammalian cells and used the protein for a phage display selection with a well-validated synthetic Fab-phage library. Two recombinant monoclonal antibodies (JLS002-rAB22 and JLS002-rAB23) were generated and validated. Their CDR sequences were determined as follows:

| Clone | CDR L1 | CDR L2 | CDR L3 | CDR H1 | CDR H2 | CDR H3 |
|---|---|---|---|---|---|---|
| JLS002- | SVSSA | SASSLYS | YAYHLI | VYSSSI | YISSYSGYTY | GRYQGM |
| rAB22 | | | | | | |
| JLS002- | SVSSA | SASSLYS | KKAYSLI | VYSSSI | SISSYSGSTS | YGYYMSGAM |
| rAB23 | | | | | | |

Both antibodies have high affinity and good expression and production in E.coli and mammalian cells. IgG and Fab (Fragment antigen-binding domain) for the two antibodies were generated. They presented good binding to CD94 and CD94-NKG2C complex expressed on Ba/F3 cells, indicating their potential as therapeutic antibodies and components in research reagents.

### 1. Fc Fusion Protein Expression and Purification

CD94 is type II membrane protein. We generated a biotinylated Fc fusion protein with the extracellular domain of CD94 fused at the C-terminal of a human IgG1 Fc (which is called “Fc-CD94” below). To produce enough of the protein that was compatible with our phage-display selection, we did optimization on the vector to improve the efficiency of protein expression and purification. In our experiments, 100-130 μg of Fc-CD94 protein with ˜90% biotinylation was purified from 30 ml of mammalian cell culture after transient transfection in Expi293cells.

### 2. Selection

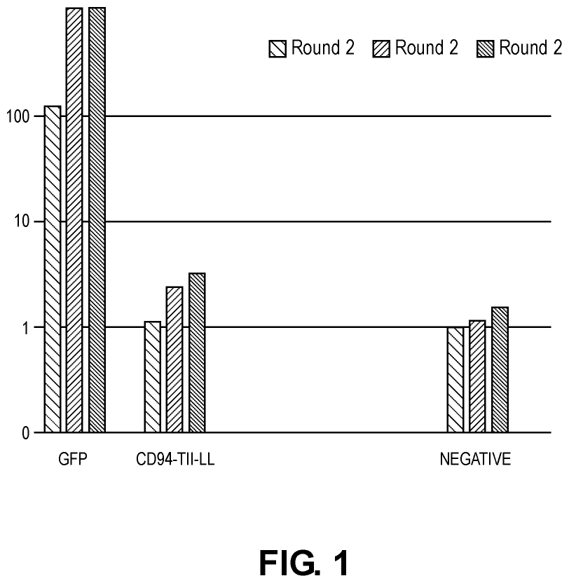

We performed direct selections against Fc-CD94 fusion protein. Enrichment for each round of the selection are shown in FIG. 1.

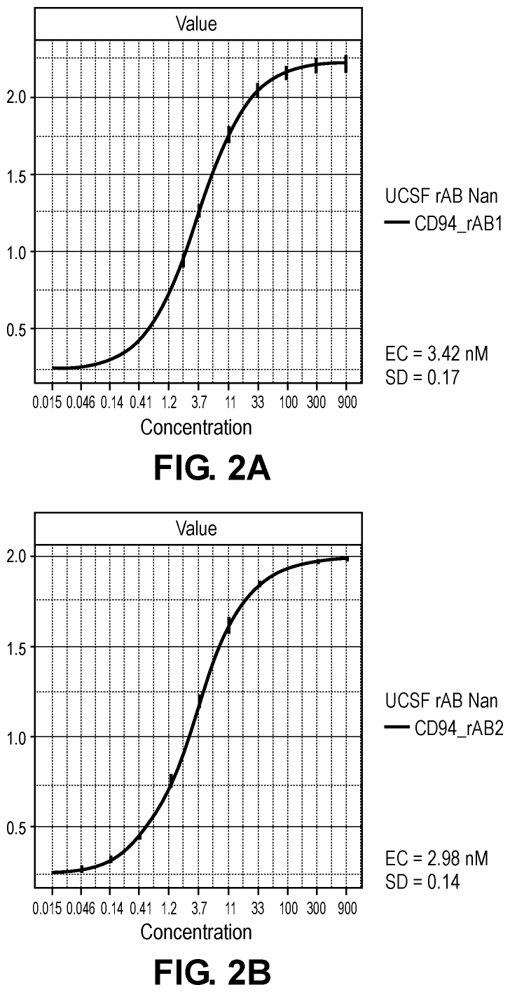

A total of 54 hits were generated from round 3 and round 4 of the selection. Two Fab phages with unique sequences were confirmed by sequencing. The two Fabs were expressed and purified. The binding affinity was determined by multipoint ELISA. The binding curves are shown in FIG. 2A-B, which indicates these two Fabs have good affinity.

### 3. Affinity of IgG

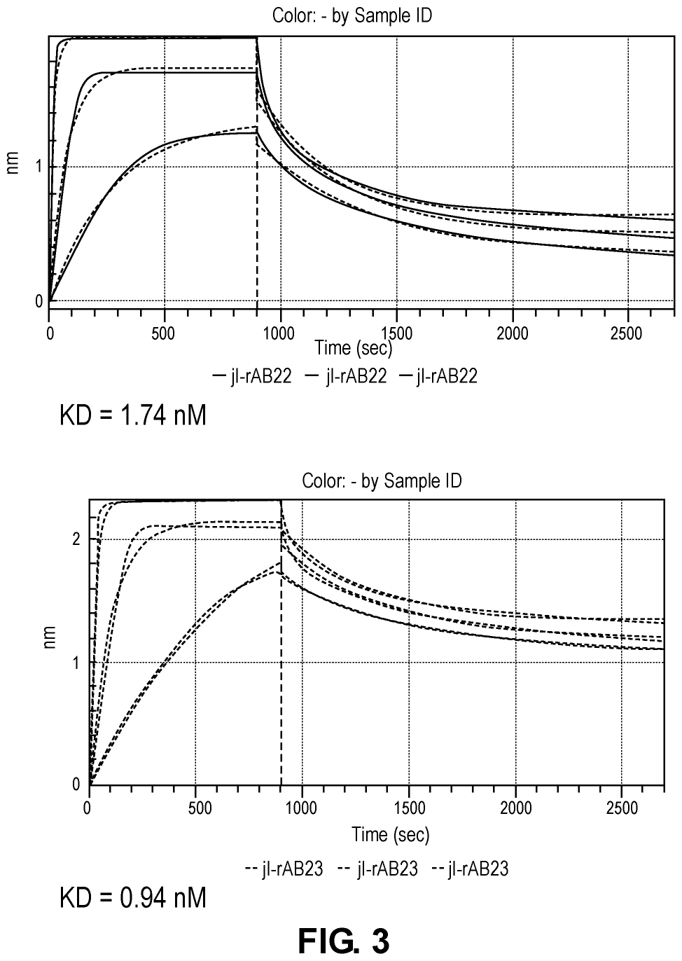

An octet assay was used to measure the affinity of IgG for the two binders JLS002-rAB22 and JLS002-rAB23. See FIG. 3. An Octet RED384 (ForteBio) instrument was used to measure the affinity of IgG for the two binders, JLS002-rAB22 and JLS002-rAB23. Fc-CD94 fusion protein was immobilized on a bio-layer interfrometry biosensor coated with streptavidin.

After blocking with PBST buffer (PBS+0.5 Tween) containing 10 μM of biotin, the IgGs being tested were loaded for 15 minutes followed by change to PBST buffer without IgG for ˜30 minutes. The kinetic binding curve was recorded.

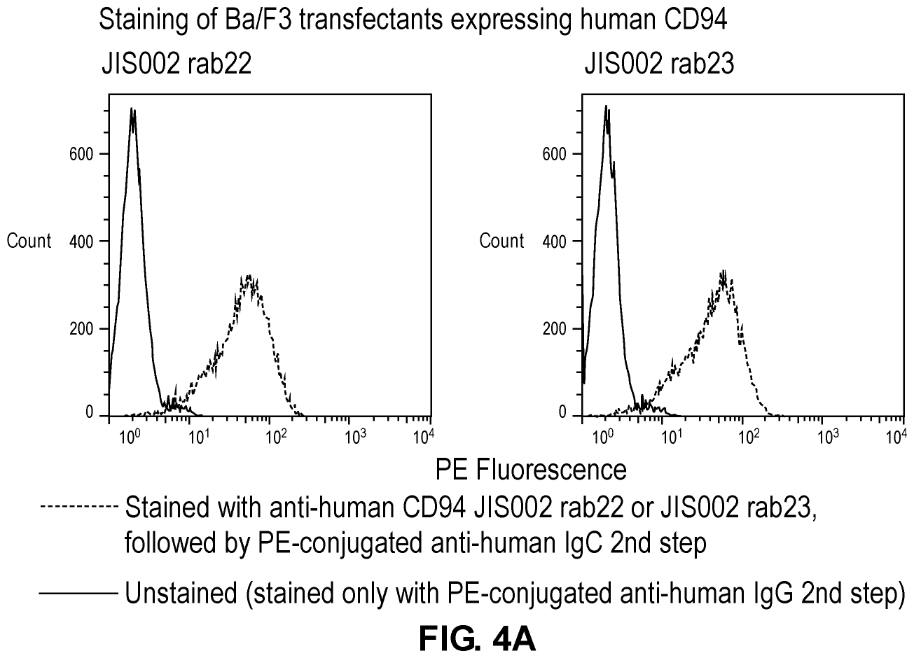

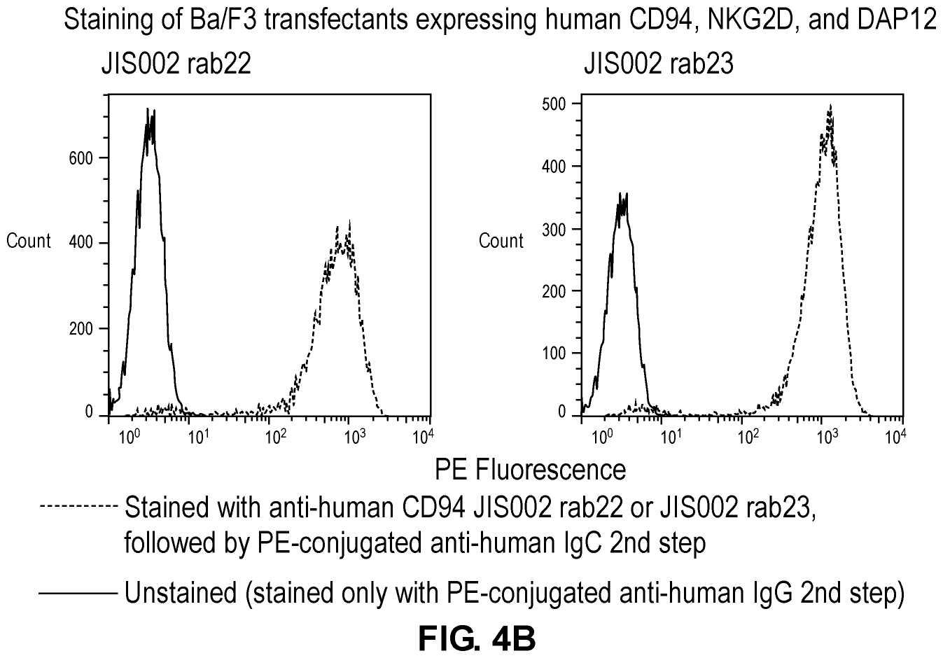

Mouse Ba/F3 cells were stably transduced with retroviral vectors encoding human CD94 or with human CD94, human NKG2C, and human DAP12 as described in Lanier, L. L., B. C. Corliss, J. Wu, and J. H. Phillips. 1998. Association of DAP12 with activating CD94/NKG2C NK cell receptors. Immunity 8:693-701. PMID: 9655483. The Ba/F3 transfectants were incubated with phosphate buffered saline (unstained) or with the rab22 JIS002 rab 22 or JSI002 rab 23 antibodies, washed, and then stained with PE-conjugated anti-human IgG1 using standard methods. The results are displayed in FIGS. 4A-B.

### Example 2

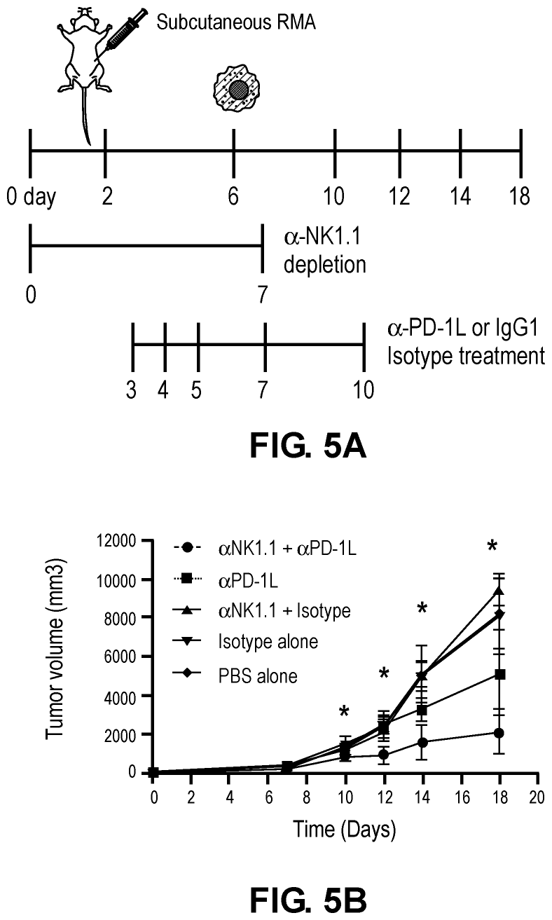

NK cell depletion was shown to improve the efficacy of α-PD-1L checkpoint blockade treatment for RMA tumor growth. C57BL/6 mice were subcutaneously administered 1×10e6 RMA lymphoma cells on day 0. As shown in FIG. 5A, the experimental design included treating mice at day 0 and day 7 with a depleting anti-NK1.1 monoclonal antibody (clone PK136), which binds to an NK cell-specific protein on mouse natural killer cells. On days 3, 4, 5, 7, and 10, α-PD-1L antibody (clone 10F.9G2) or control rat IgG2b isotype-matched antibodies were administered to the mice. Intraperitoneal injection of α-NK1.1, α-PD-1L, or IgG2b isotype-matched control antibody in amounts of 150 μg, 250 μg, and 250 μg in saline were injected at the indicated time points. Measurements of tumor volume over time for mice under the various treatments. FIG. 5B provides a graph of the resulting data, showing that depletion of NK cells with an anti-NK cell antibody significantly reduced tumor volumes compared to controls in the checkpoint blockade α-PD-1L antibody-treated mice. Significance is noted as * P<0.05 by Student's t test.

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1. A method of enhancing a T cell response in an individual in need thereof, comprising administering an anti-natural killer cell-specific antibody to the individual in an amount sufficient to delete natural killer cells.

2. The method of claim 1, wherein the natural killer cell-specific antibody is an anti-CD94 specific antibody.

3. The method of claim 1, wherein the individual has cancer and activated T cells in the individual target cancer cells in the individual.

4. The method of claim 1, further comprising before or after or simultaneous with administering the anti-CD94 specific antibody to the individual, administering one or more checkpoint inhibitor.

5. The method of claim 4, wherein the checkpoint inhibitor is an anti-CTLA-4, anti-PD-1, or anti-PD-L1 antibody.

6. The method of claim 2, wherein the antibody comprises:
a heavy chain variable region comprising a heavy chain complementarity-determining region (CDR) 1, CDR2, and CDR3 of SEQ ID NO:1, SEQ ID NO:2, and SEQ ID NO:3, respectfully; and
a light chain variable region comprising a light chain CDR1, CDR2, and CDR3 of SEQ ID NO: 4, SEQ ID NO:5, and SEQ ID NO:6, respectfully; or
a heavy chain variable region comprising a heavy chain CDR1, CDR2, and CDR3 of SEQ ID NO:7, SEQ ID NO:8, and SEQ ID NO:9, respectfully; and
a light chain variable region comprising a light chain CDR1, CDR2, and CDR3 of SEQ ID NO: 10, SEQ ID NO:11, and SEQ ID NO: 12, respectfully.

7. The method of claim 1, wherein the antibody is a chimeric antibody.

8. The method of claim 7, wherein the chimeric antibody is a humanized antibody.

9. The method of claim 1, wherein the antibody is a human or synthetic antibody.

10. The method of claim 1, wherein the antibody comprises:
a heavy chain variable region comprising SEQ ID NO: 13 and a light chain variable region comprising SEQ ID NO: 14; or
a heavy chain variable region comprising SEQ ID NO:15 and a light chain variable region comprising SEQ ID NO: 16.

11. The method of claim 1, wherein the antibody induces antibody-dependent cellular cytotoxicity (ADCC) or antibody-dependent phagocytosis (ADP).

12. The method of claim 1, wherein the antibody is linked to a toxin.

13. A method of improving the survival of autologous or allogeneic cells transplanted into an individual, the method comprising:
administering an anti-natural killer cell-specific antibody to the individual, who receives the transplanted cells before, after or during the administering, in an amount sufficient to transiently deplete natural killer cells.

14. The method of claim 13, wherein the natural killer cell-specific antibody is an anti-CD94 specific antibody.

15-17. (canceled)

18. The method of claim 13, wherein the individual has cancer.

19. The method of claim 14, wherein the antibody comprises:
a heavy chain variable region comprising a heavy chain complementarity-determining region (CDR) 1, CDR2, and CDR3 of SEQ ID NO:1, SEQ ID NO:2, and SEQ ID NO:3, respectfully; and
a light chain variable region comprising a light chain CDR1, CDR2, and CDR3 of SEQ ID NO: 4, SEQ ID NO:5, and SEQ ID NO:6, respectfully; or
a heavy chain variable region comprising a heavy chain CDR1, CDR2, and CDR3 of SEQ ID NO:7, SEQ ID NO:8, and SEQ ID NO:9, respectfully; and
a light chain variable region comprising a light chain CDR1, CDR2, and CDR3 of SEQ ID NO: 10, SEQ ID NO:11, and SEQ ID NO: 12, respectfully.

20-37. (canceled)

38. A monoclonal antibody that binds CD94, the antibody comprising:
a heavy chain variable region comprising a heavy chain complementarity-determining region (CDR) 1, CDR2, and CDR3 of SEQ ID NO:1, SEQ ID NO:2, and SEQ ID NO:3, respectfully; and
a light chain variable region comprising a light chain CDR1, CDR2, and CDR3 of SEQ ID NO: 4, SEQ ID NO:5, and SEQ ID NO:6, respectfully; or
a heavy chain variable region comprising a heavy chain CDR1, CDR2, and CDR3 of SEQ ID NO:7, SEQ ID NO:8, and SEQ ID NO:9, respectfully; and
a light chain variable region comprising a light chain CDR1, CDR2, and CDR3 of SEQ ID NO: 10, SEQ ID NO:11, and SEQ ID NO: 12, respectfully.

39. The monoclonal antibody of claim 38, wherein the antibody is a chimeric antibody.

40. The monoclonal antibody of claim 38, wherein the chimeric antibody is a humanized antibody.

41. The monoclonal antibody of claim 38, wherein the antibody comprises:
a heavy chain variable region comprising SEQ ID NO:13 and a light chain variable region comprising SEQ ID NO:14; or
a heavy chain variable region comprising SEQ ID NO:15 and a light chain variable region comprising SEQ ID NO:16.
